(19) United States
(12) Patent Application Publication
(10) Pub. No.: US 2025/0009972 A1
(43) Pub. Date: Jan. 9, 2025

(54) SYSTEMS, DEVICES, AND METHODS FOR FLUIDIC THROTTLE CONTROL OF THE DELIVERY OF THERAPIES IN NEEDLE-BASED DELIVERY SYSTEMS

(71) Applicant: Rx Bandz, Inc., Locust Valley, NY (US)

(72) Inventors: Stephen HARHEN, Rumson, NJ (US); Edward Paul HARHEN, Duxbury, MA (US)

(73) Assignee: Rx Bandz, Inc., Locust Valley, NY (US)

(21) Appl. No.: 18/779,996

(22) Filed: Jul. 22, 2024

Related U.S. Application Data
(63) Continuation of application No. PCT/US2023/061093, filed on Jan. 23, 2023.
(60) Provisional application No. 63/412,109, filed on Sep. 30, 2022.
(60) Provisional application No. 63/301,864, filed on Jan. 21, 2022.

(51) Int. Cl. A61M 5/20; A61M 5/158; A61M 5/315; A61M 5/32

(52) U.S. Cl. CPC A61M 5/2053; A61M 5/158; A61M 5/31578; A61M 5/3159; A61M 5/3221; A61M 5/3287; A61M 2005/1585; A61M 2005/2006; A61M 2005/206; A61M 2205/123; A61M 2205/19; A61M 2205/273; A61M 2205/3331; A61M 2205/82; A61M 2206/10

(57) ABSTRACT

Systems, devices, and methods described herein relate to delivery of therapeutic agents. Such systems and devices can include needle-based delivery systems, in which a throttling assembly is configured to control a rate or needle insertion and/or a rate of drug extrusion.

### CROSS-REFERENCE TO RELATED APPLICATIONS

This application is a continuation of International Patent No. PCT/US2023/061093, filed Jan. 23, 2023, and titled “SYSTEMS, DEVICES, AND METHODS FOR FLUIDIC THROTTLE CONTROL OF THE DELIVERY OF THERAPIES IN NEEDLE-BASED DELIVERY SYSTEMS,” which claims priority to pending U.S. Provisional Patent Application No. 63/412,109, filed Sep. 30, 2022, and titled “SYSTEMS, DEVICES, AND METHODS FOR FLUIDIC THROTTLE CONTROL OF THE DELIVERY OF THERAPIES IN NEEDLE-BASED DELIVERY SYSTEMS,” and U.S. Provisional Patent Application No. 63/301,864, filed Jan. 21, 2022, and titled “FLUIDIC THROTTLE CONTROL FOR THE DELIVERY OF STANDARD AND RHEOLOGICALLY CHALLENGING THERAPIES IN NEEDLE-BASED DRUG DELIVERY SYSTEMS,” the disclosures of each of which are incorporated by reference in their entireties.

### TECHNICAL FIELD

Systems, devices, and methods described herein generally relate to fluidic throttle control for the delivery of therapeutic substances in needle-based delivery systems. In some embodiments, the present disclosure relates to the delivery of standard therapies, while in other embodiments, the present disclosure relates to the delivery of rheologically challenging therapies, such as viscous, shear sensitive, or non-Newtonian therapies. A needle-based delivery system can be used to deliver such therapies. In some embodiments, the needle-based delivery system can include a fluidic throttle that interacts with an intermediary fluid. The fluidic throttle can act as a rate limiter, providing a throttled rate and constant force of drug extrusion, regardless of the force applied by the stored energy system. The fluidic throttle can also enable different forces to be applied for controlling needle ejection and drug extrusion.

### BACKGROUND

Devices have been used to administer therapies under emergency conditions, such as, for example, administering epinephrine to counteract the effects of a severe allergic reaction (e.g., anaphylaxis) or naloxone for opioid overdose. Devices have also been described for use in administering therapies to treat disease, such as, for example, anti-arrhythmic medications and selective thrombolytic agents during a heart attack. One common type of delivery device is an auto-injector. Auto-injectors offer an alternative to syringes manually operated by patients for administering therapeutic agents into subjects in need thereof by allowing subjects in need thereof to self-administer medications.

Auto-injectors can be the preferred method of delivery for many therapeutics due to their ease of use by both trained and untrained professionals. Additionally, the speed of delivery and dose accuracy can be desired for quick administration. With auto-injectors, a small needle may be desirable to reduce pain associated with needle insertion. However, the small inner diameter and length of a needle acts to create a restricted fluid flow, which is an undesirable condition that becomes more restrictive as needle inner diameter size decreases. This limiting condition acting on the fluid flow through the restrictive needle can limit or prevent fluid flow of a therapy. The slowdown or stalling of the fluid in the needle can be exacerbated in viscous, shear sensitive or non-Newtonian pharmaceutical therapies, such as proteins and monoclonal antibodies.

In attempts to overcome the restrictive inner diameter of the needle, higher pressures may be applied. Such higher pressures, however, may generate high pressures in the primary drug container and in the needle, which can increase the risk of cartridge rupture or stack clogging in the needle. This is a phenomenon that is inherent in needle-based injection systems, because the needle acts as the primary restrictive orifice. High initial forces can result in blockage or exponential increases in apparent viscosity of a therapy to be extruded, due to the needle acting as a block in the system. This can result in undelivered or, in some cases, a damaged therapeutic due to shear forces. Compared to standard therapies, rheologically-challenging therapies can present more challenges. For example, such therapies can require higher forces to extrude the therapy through small needles, but are also more likely to clog in the needles. As an alternative, larger needles in conjunction with less powerful energy systems can be used, but while such needles may avoid clogging due to restricted flow, they can cause more patient discomfort, do not have the power to deliver larger volume doses, and cannot regulate dose over time.

To provide high enough pressures that can overcome stalling given a small needle, a powerful single energy force, such as a spring, may be used to deploy a therapy. The forces generated by these powerful energy sources, however, can exacerbate the risk of system failure. Specifically, the high speeds and impact forces experienced during auto-injector activation can result in mechanical damage on the surface of a cartridge, such as a glass cartridge, which can fracture, as the cartridge can come into contact with a needle hub, interior components, and/or interior walls of the auto-injector.

Alternatively, multiple or variable stored energy systems can be used, but these add significant complexity to the delivery system design. This added complexity can fail to meet regulated reliability demands. In particular, needle ejection, dose accuracy, and time to deliver a therapy are required to have a reliability of 99.99%. Multiple energy sources, such as a two-spring system with one spring to eject the needle and another to extrude the drug, add to the complexity and may reduce reliability to unacceptable levels. Therefore, there remains a need for a drug delivery system that can meet current reliability standards and avoid the downsides of existing single stored energy delivery systems.

### BRIEF DESCRIPTION

Systems, devices, and methods described herein relate to needle-based injection systems including fluidic throttle elements for the delivery of therapeutic substances. Such delivery systems can have advantages over manual drug delivery systems (e.g., manually actuated syringes) and advantages over traditional auto-injectors that use a single stored energy system. In some embodiments, delivery systems described herein may use a intermediary fluid (e.g., a hydraulic or pressurized fluid) that interacts with a fluidic throttle to deliver a therapeutic fluid that is located within a drug container (e.g., a glass cartridge). The fluidic throttle may enable a single stored energy device to be used to eject a needle and to deliver a therapeutic fluid, without the risk of generating forces that would result in failures during delivery.

In some embodiments, a drug delivery system can be an auto-injector that provides a driving force source (e.g., spring, liquid or gas fluids, or other mechanism(s)), which apply pressure to a plunger of a drug container (e.g., a glass cartridge). The drug container can include a glass container, a crimp cap with a seal, and the plunger. Initially, the drug container can be driven forward toward a needle sub-assembly, puncturing the seal and/or a needle sheath and driving the needle into the patient. The plunger, internal to the glass container, can then be driven forward to extrude a therapeutic fluid located within the drug container. In drug delivery systems without a fluidic throttle, the rate that the needle is driven at and the rate of drug extrusion can be the same, e.g., as a result of being driven by the same driving force of a stored energy device. In such systems, the flow of the therapeutic fluid is rate restricted by needle fluid flow characteristics.

Given the drawbacks of existing needle-based drug delivery systems, there is a continuing need for a throttle using an intermediary fluid to replace the needle as the fluid restriction point and enables an extrusion rate control that provides a constant drug extrusion rate. Gas pressure driven systems suffer from effects of temperature change as well as pressure decrease as the gas is exhausted. Spring driven systems suffer from force reduction as the spring force is released. A throttle as disclosed herein can directly control, and maintain consistently, the desired internal pressure in a drug container and therefore a rate of drug extrusion, e.g., as driven by a stored energy component. The throttle can allow the stored energy component to apply sufficient pressure to a plunger of a drug container while avoiding drug stalling in the needle or rupture of the drug container. The throttle can work in tandem with a working or intermediary fluid having desired densities and/or vapor pressures to provide a constant transfer of force from the stored energy component to the drug cartridge.

Therapy or drug delivery systems as described herein enable one to separate the force driving needle insertion from the force driving a cartridge plunger. Embodiments of the present technology specifically apply to drug delivery devices that use a single stored energy source with an intermediary fluid flowing through a throttle before being applied to a plunger for extrusion of therapeutic fluid. A cartridge, such as a glass container, typically includes a bottom heel at the base of a barrel that extends to a shoulder and a neck. In embodiments, the bottom heel of the cartridge can be fitted with a fluidic throttle, which can include an orifice configured to modulate a speed of fluid flow, e.g., to thereby modulate a pressure applied to the cartridge plunger. The throttle on the bottom heel of the cartridge therefore acts as a throttle controlling the flow of fluid to the cartridge plunger and thereby controlling the velocity of the plunger and the rate of drug extrusion. Throttling in this case is associated with a choked condition or choked flow, as commonly associated with the venturi effect. Choking occurs when a working fluid either approaches the speed of sound or changes phase. Under such conditions, the flow rate does not increase with an increase in driving pressure. In some embodiments, the throttle can also define an effective piston size for driving a needle forward. Since the throttle is coupled to the bottom heel of the cartridge, a surface area of the backside of the throttle (or outer diameter of the backside of the throttle, in the case of a circularly shaped throttle backside) can form the effective piston size for driving the entire cartridge and therefore the needle forward. This backside surface area of the throttle and the configuration of the throttle orifice allow for separate and discrete modulation of the rate of drug extrusion and the rate or force of needle ejection.

In some embodiments, the intermediary fluid or drive fluid can be either liquid or gaseous, e.g., with vapor points and density designed to work in tandem with the throttle to maintain pressure applied to the drug container. In particular, the intermediary fluid and the throttle orifice work in tandem to prevent the needle from acting as the restrictive orifice in the system.

The throttle may be positioned at any point between the stored energy device and the plunger of a drug container or cartridge. In some embodiments, the throttle can be positioned on a bottom heel of the drug cartridge, while in other embodiments, the throttle can be positioned separately or apart from the drug cartridge. When positioned over the drug cartridge, the throttle may or may not contact the inner surface of the drug cartridge. In some embodiments, the throttle can have an initial inner diameter such that it can be sleeved over the bottom heel of a drug cartridge and a recovered inner diameter to secure itself on the cartridge. In some embodiments, the throttle can be coupled to the drug cartridge, e.g., by chemically adhering the throttle to the cartridge. In some embodiments, the throttle can be under compression to secure the throttle around at least a portion of the drug cartridge.

In some embodiments, an apparatus includes: a needle having a proximal end and a distal end; a cartridge having a body, a plunger, and a seal, the body, the plunger, and the seal collectively defining a reservoir configured to contain a therapeutic agent; a stored energy device configured to apply pressure to an intermediary fluid; a throttling assembly disposed on the body of the cartridge upstream of the plunger, the throttling assembly including a restrictor configured to limit a flow of the intermediary fluid therethrough; and an activation device configured to activate the stored energy device to apply pressure to the intermediary fluid such that the intermediary fluid acts on the throttling assembly to: initially drive a movement of the cartridge and the needle to insert the distal end of the needle into tissue and the proximal end of the needle through the seal and into the reservoir; and subsequently drive a movement of the plunger to extrude the therapeutic agent out through the needle.

In some embodiments, a method includes activating, by pressing an activation device, a stored energy device of a therapeutic delivery system; in response to activating the stored energy device, applying pressure on an intermediary fluid to cause the intermediary fluid to act on a throttling assembly of the therapeutic delivery system; in response to the intermediary fluid acting on the throttling assembly, driving movement of a cartridge and a needle of the therapeutic delivery system toward the skin of a patient to insert the needle into the skin; in response to the intermediary fluid acting on the throttling assembly, driving movement of the cartridge toward the needle such that the needle punctures through a seal of the cartridge to be in fluid communication with a therapeutic agent within the cartridge; in response to the intermediary fluid acting on the throttling assembly, generating a throttled flow of intermediary fluid via the throttling assembly; and driving movement of a plunger of the cartridge to extrude an amount or volume of the therapeutic agent in response to the throttled flow of the intermediary fluid acting on the plunger.

In some embodiments, an apparatus includes: a cartridge having a plunger and a reservoir configured to contain a therapeutic agent; a needle configured to extrude the therapeutic agent; a stored energy device configured to apply pressure to an intermediary fluid; a throttling assembly disposed upstream of the plunger in a pathway of the intermediary fluid; and an activation device configured to activate the stored energy device to apply pressure to the intermediary fluid such that the throttling assembly generates a flow of the intermediary fluid that applies a constant pressure on the plunger to extrude the therapeutic agent through the needle.

### DRAWINGS

The drawings described herein are for illustrative purposes only of selected embodiments and not all possible implementations, and are not intended to limit the scope of the present disclosure.

FIG. 1 schematically depicts an example therapeutic delivery system, according to embodiments.

FIG. 2 schematically depicts an example throttling element of a therapeutic delivery system, according to embodiments.

FIGS. 3A-3D schematically depict the operation of a therapeutic delivery system, at different stages of operation, according to embodiments.

FIG. 4 depicts a flow chart of an example method of operating a therapeutic delivery system, according to embodiments.

FIGS. 5A and 5B depict a side view and a cross-sectional view of a cartridge and a needle hub assembly, respectively, according to embodiments. FIG. 5B depicts the cross-section along section line 5B as shown in FIG. 5A.

FIGS. 6A and 6B depict a side view and a cross-sectional view of a cartridge and a needle hub assembly, respectively, according to embodiments. FIG. 6B depicts the cross-section along section line 6B as shown in FIG. 6A.

FIGS. 7A-7C depict different views of an example throttling element, according to embodiments.

FIGS. 8A and 8B depict a side view and a cross-sectional view of a cartridge and a needle hub assembly, respectively, with the cartridge fitted with a throttling element, according to embodiments. FIG. 8B depicts the cross-section along section line 8B as shown in FIG. 8A.

FIGS. 9A and 9B depict example throttling elements, with different restricted flows, according to embodiments.

FIGS. 10A and 10B depict a side view and a cross-sectional view of a cartridge fitted with a throttling element, respectively, where a proximal end of the throttling element is configured for initial needle ejection, according to embodiments. FIG. 10B depicts the cross-section along section line 10B as shown in FIG. 10A.

FIG. 11 is a perspective view of an example therapeutic delivery system, according to embodiments.

FIGS. 12A-12F depict the operation of a therapeutic delivery system, at different stages of operation, according to embodiments.

FIG. 13 is a graphical representation of an intermediary fluid flow rate through a restrictive orifice of a throttling element, according to embodiments.

### DETAILED DESCRIPTION

Systems, devices, and methods are described herein for delivering a therapeutic substance to a patient, e.g., via a needle-based delivery system such as an auto-injection system.

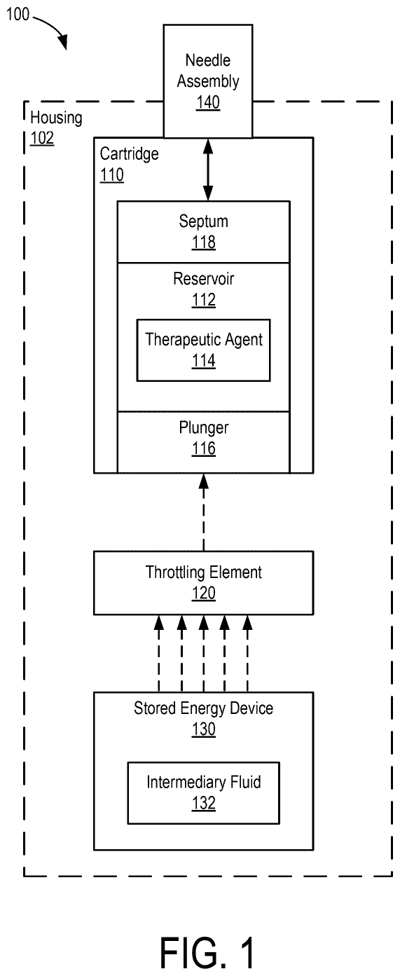

As schematically illustrated in FIG. 1, a therapeutic delivery system 100 can include a needle assembly 140, a cartridge 110, a throttling element or assembly 120, and a stored energy device 130, according to embodiments. Optionally, the therapeutic delivery system 100 can include a housing 102 that houses or contains one or more of the needle assembly 140, the cartridge 110, the throttling element 120, and the stored energy device 130. The therapeutic delivery system 100 can be any type of needle-based system or device for delivering a dose (e.g., an amount or volume) of a therapeutic substance. In some embodiments, the therapeutic delivery system 100 can be an auto-injector or auto-injection system, e.g., such as a device that is self-administered by a patient who is experiencing an emergency health condition. In some embodiments, the therapeutic delivery system 100 can be a miniaturized wearable injection device. In some embodiments, the therapeutic delivery system 100 can be configured to be a handheld device or be integrated into a handheld device.

The housing 102 can include one or more sections. In some embodiments, the housing 102 can include a main body and a removable cap that is configured to cover at least a portion of the main body. The removable cap can be configured to cover the portion of the main body so that a needle contained within the main body is shielded, e.g., to maintain sterility of the needle prior to use and/or to avoid accidental needle ejection. In some embodiments, the removable cap can also be placed over the main body such that an activation device or actuator for activating the therapeutic delivery system 100 is covered, e.g., to avoid accidental activation. In use, the removable cap of the housing 102 can be removed to enable the needle to be ejected into a patient's body. In some embodiments, the portion of the main body that is exposed once the removable cap is removed can be pressed or placed against a tissue surface, and an activation device (e.g., a button, switch, slider, knob, etc.) can be activated to cause the needle to eject out of the main body and through the tissue surface.

The needle assembly 140 can include a needle or cannula and a needle hub. The needle can include a sharp distal end that extends from the hub and is configured to penetrate through tissue. The needle hub can support the needle, e.g., relative to the cartridge 110, housing 102, and/or other portions of the therapeutic delivery system 100. In some embodiments, the needle may be held within the housing 102 (e.g., held within a main body of the housing 102) until an activation element is activated (e.g., a button, switch, or other activation device is pressed or actuated). In some embodiments, the needle can be a double-ended needle and include a proximal end that is also sharp. In use, the proximal end of the needle can be configured to puncture through a septum, membrane, or other seal 118.

The cartridge 110 can include a septum 118, a reservoir 112 that contains a therapeutic agent 114, and a plunger 116. In some embodiments, the cartridge 110 can include a body that is formed of a rigid material, such as, for example, glass, plastic, metal, or some combination of such materials. For example, the cartridge 110 can include a glass container, e.g., formed of borosilicate glass. In some embodiments, the body can include a bottom heel and a barrel that extends to a shoulder and a neck. The body can have an inner surface and an outer surface, with a wall thickness extending between the inner and outer surfaces. The reservoir 112 can be bounded on one end by the septum 118 and on the other end by the plunger 116. When not in use, the septum 118, plunger 116, and body of the cartridge 110 contain the therapeutic agent 114 within the reservoir 112.

The therapeutic agent 114 can include at least one of a drug, a vaccine, a protein, a peptide, a gene, a compound or another pharmaceutically active ingredient. Examples of therapeutic agents suitable for use in the system 100 include glucagon, insulin, adrenaline, epinephrine, anti-venom, atropine, antibody formulations, antidotes to chemical agents, and the like. In some embodiments, the medication suitable for use in the device of the present invention is at least one medication selected from the group of medications identified by tradenames consisting of Acthar, Actimmune, Apokyn, AquaMephyton, Aranesp, Arixtra, Avonex, Betaseron, Bravelle, Butorphanol, Byetta, Calcijex, Calcitonin, Caverject, Cetrotide, Chorionic Gonadotropin, Cimzia, Copaxone, Copegus, DDAVP, D.H.E-45, Delatestryl, Delestrogen, Depo-Estradiol, Depo-Provera 150, Depo-SubQ Provera 104, Depo-Testosterone, Desmopressin, Dihydroergotamine, Edex, Eligard, Enbrel, Epipen, Epogen, Exjade, Faslodex, Fertinex, Follistim, Forteo, Fragmin, Fuzeon, Ganirelix acetate, Genotropin, Gleevec, Glucagon, Gonal, Heparin, Humatrope, Humira, Imitrex, Increlex, Infergen, Innohep, Insulin, Intron A, iPlex, Ketorolac, Kestrone, Kineret, Kuvan, Leukine, Leuprolide Acetate, Lovenox, Lupron, Luveris, Medroxyprogesterone, Menopur, Methotrexate, Miacalcin, Muse, Neumega, Neulasta, Neupogen, Nexavar, Norditropin, Novarel, Nutropin, NuvaRing, Omnitrope, Orfadin, Ovidrel, Pegasys, Peg-Intron, Pregnyl, Procrit, Profasi, Progesterone, Pulmozyme, Raptiva, Rebetol, Rebif, Repronex, Revlimid, Ribasphere, Ribavirin, Saizen, Sandostatin, Sensipar, Serostim, Somatuline, Sprycel, Somavert, Stadol, Sumatriptan, Supprelin, Sutent, Symlin, Tarceva, Testosterone, Temodar, Tev-Tropin, Thalomid, Tobi, Tykerb, Vitamin B12, Vitamin D, Vitamin K, Xeloda, Zemplar, and Zorbtive. In some embodiments, the system 100 is capable of delivering highly viscous therapeutic agents (e.g., having a kinetic viscosity greater than that of water, having a kinetic viscosity greater than 30 centipoise, having a kinetic viscosity between 30 centipoise and 1500 centipoise, etc.), such as biologicals, flowable tissues, connective tissue matrixes or monoclonal antibodies. It will be apparent to those of skill in the art that the therapeutic agents listed above are only exemplary and that the system 100 is equally suitable for use in connection with practically any other injectable therapeutic agent not specifically listed herein.

The septum or seal 118 can function as a partition, enclosing or sealing off the therapeutic agent 114 from an external environment. In some embodiments, septum 118 can be implemented as a crimp cap seal or septum (e.g., a crimp camp combi-seal) that is to the body of the cartridge 110. The septum 118 can be formed of a flexible material, e.g., silicone, rubber, or other type of elastomer. The septum 118 can be disposed at a distal or dispensing end of the cartridge 110. The dispensing end of the cartridge 110 can be configured to receive a needle hub of the needle assembly 140 thereon, such as, for example, the needle hub of a double-ended needle. The needle hub can be coupled to the dispensing end by any known technique, such as threaded connection or snap fit configurations. Once advanced onto the cartridge 110, the double-ended needle can pierce the septum 118 to dispense the therapeutic agent 114 from the cartridge 110.

In use, one end of a needle of the needle assembly 140 can be placed within the reservoir 112, e.g., after puncturing through the septum 118, and another end of the needle can be inserted into a patient's body. Fluid communication of the therapeutic agent 114 can then be established through the needle into the patient's body. The plunger 116 can slide along a length of the body of the cartridge 110, e.g., by sliding along the inner surface of the body of the cartridge 110. As the plunger 116 moves distally toward the needle, the plunger 116 can extrude an amount or volume of the therapeutic agent 114 through the needle and into the patient's body. The amount or volume extruded can be a predetermined or preset amount, e.g., dependent on the therapy or treatment being delivered to the patient.

The plunger 116 can be driven by energy imparted by a stored energy device 130. In some embodiments, the plunger 116 can be driven hydraulically, e.g., by an intermediary fluid 132 (e.g., a hydraulic or pressurized liquid and/or gas). Alternatively or additionally, the plunger 116 can be driven manually, e.g., via a user pressing down on a shaft, slider, or other mechanical actuator that is coupled to the plunger 116. Still alternatively or additionally, the plunger 116 can be driven mechanically, e.g., using a spring.

The stored energy device 130 can be configured to supply energy, e.g., to drive movement of the needle, the plunger 116, or other components of the therapeutic delivery system 110. In some embodiments, the stored energy device 130 can be configured to supply energy in response to being released or activated. For example, an activation device such as a button, tab, slider, knob, or other mechanism can be coupled to the stored energy device 130, and upon actuation of such device, the stored energy device 130 can be configured to deliver stored energy to drive the delivery of the therapeutic agent 114. In some embodiments, the stored energy device 130 can include a power or deployment spring that drives a piston, which then delivers intermediary fluid 132 to the plunger 116. The deployment spring can be locked in a compressed configuration, and can be released upon actuation of an activation device. Alternatively, in some embodiments, the stored energy device 130 can include an electric motor to drive the piston. Still alternatively, in some embodiments, the stored energy device 130 can include pressurized fluid containers that can open to release an intermediary fluid 130. It can be appreciated that other suitable forms of energy storage and release can be used with the stored energy device 130 without departing from the scope of the present disclosure.

In some embodiments, the energy provided by a single stored energy device 130 can be used to drive needle ejection or insertion (e.g., via movement of the needle assembly 140) and to drive drug extrusion (e.g., via movement of the plunger 116). Alternatively, multiple stored energy devices 130 can be used, with each stored energy device 130 being used to drive a different component of the therapeutic delivery system 100, serially or in parallel. For example, a first stored energy device 130 can be configured to supply energy for driving needle ejection, while a second stored energy device 130 can be configured to supply energy for driving drug extrusion or plunger movement.

In some embodiments, a throttling element 120 can be used to restrict a portion of the flow of intermediary fluid 132 to the plunger 116. The throttling element 120 can be disposed between a source of the intermediary fluid 132 and the plunger 116. In some embodiments, the throttling element 120 can be coupled to the cartridge 110. For example, the throttling element 120 can extend over a bottom heel of the cartridge 110. In some embodiments, the throttling element 120 can be formed of one or more medical grade elastomers, such as, for example, liquid silicone rubber (LSR), fluoroelastomers (FKM), perfluoroelastomers (FFKM), and ethylene-propylene diene monomer (EPDM). In some embodiments, the throttling element 120 can have a minimum Shore hardness of 40A.

The throttling element 120 can be configured to replace the needle as the fluid rate limiting orifice in a hydraulically driven therapeutic delivery system. In particular, the throttling element 120 can include an opening or orifice having a small cross-sectional area, thereby limiting the fluid flow of the intermediary fluid 132 to the plunger 116 and the rate of drug extrusion. In some embodiments, the throttling element 120 can include a single opening having a circular cross-sectional area. In some embodiments, the circular cross-sectional area can have a diameter of less than about 5 mm, less than about 4 mm, less than about 3 mm, less than about 2 mm, less than about 1 mm, or less than about 0.5 mm, including all values and sub-ranges therebetween (e.g., between about 0.5 mm and about 5 mm, or between about 0.5 mm and about 2 mm). In some embodiments, throttling element 120 and include multiple openings. In some embodiments, the multiple openings can have a combined cross-sectional area that is equal to the cross-sectional area of a single opening having a circular cross-sectional area with a diameter of less than about 5 mm, less than about 4 mm, less than about 3 mm, less than about 2 mm, less than about 1 mm, or less than about 0.5 mm, including all values and sub-ranges therebetween. In some embodiments, the throttling element 120 can include a single or multiple openings with different cross-sectional shapes, e.g., circular, oval, square, rectangular, etc.

By replacing the needle as the rate limiting orifice, the throttling element 120 can prevent or mitigate pressures during therapeutic delivery that can result in a blockage in the needle and/or breakage of the cartridge 110, especially with a more viscous, shear sensitive, or non-Newtonian fluid. The throttling element 120 also serves as an intermediary component between the stored energy device 130 and the cartridge 110, which can alleviate direct forces being applied to the cartridge. In some embodiments, the throttling element 120 can be of monolithic construction. Alternatively, the throttling element 120 can be formed of multiple parts, e.g., which can be joined together during assembly.

The restrictive orifice or opening of the throttling element 120 can be varied, e.g., to control the rate of drug extrusion. For example, the restrictive orifice of the throttling element 120 can be reduced to reduce the rate of drug extrusion below the restrictive limitations of a given needle gauge. The throttling element 120 can be configured to provide a flowrate for the intermediary fluid that remains constant or substantially constant, which in turn allows for constant or substantially constant therapeutic delivery rates. As such, the therapy delivery time can be controlled by adjusting the restrictive orifice of the throttling element 120. In some embodiments, the intermediary fluid can be a Newtonian fluid, such as, for example, water, oil, alcohol, or combination thereof. In some embodiments, the intermediary fluid can be a non-Newtonian fluid, such as a slurry, a gel, a colloid, etc.

In some embodiments, the system 100 including the throttling element or assembly 120 can be for discretely and/or separately controlling the forces applied to the cartridge during forward advancement and needle insertion and the forces applied to the plunger for drug extrusion using a single stored energy device (e.g., power spring). The throttling assembly is configured to allow for the rate of insertion of the needle into the tittle (due to the pressure applied to the cartridge and needle hub assembly) to be greater or less than the pressure applied to on the plunger of the cartridge to determine the rate of drug extrusion. The purpose being to protect the cartridge from initial high force impacts or high internal pressures that are generated from high initial autoinjector spring forces, while still being able to generate pressure required to extrude a therapeutic substance such as a viscous drug.

While a single throttling element 120 and a single cartridge 110 are depicted in FIG. 1, it can be appreciated that the system 100 can include multiple throttling elements 120 and/or cartridges 110, e.g., for delivery of multiple therapies. For example, in some embodiments, a single stored energy device 130 can be used to deliver multiple therapeutic agents, which are contained in different cartridges 110. In such embodiments, multiple throttling elements 120 with the same or different restrictive orifices can be used to control the rate of extraction of each therapeutic agent. The different restrictive orifices of the throttling elements 120 can be selected based on the type of therapeutic agent being delivered (e.g., standard vs. viscous), the gauge of the needle delivering that therapeutic agent, and/or the timing of when that therapeutic agent is to be delivered. For example, a first throttling element 120 may have a more restrictive orifice (e.g., an orifice with a smaller cross-sectional area) than a second throttling element 120, where the first throttling element 120 is being used for the delivery of a non-Newtonian fluid compared to the second throttling element 120.

In some embodiments, certain components of needle-based injection systems as described herein can be similar to those described in U.S. Patent Application Publication No. 2021/0386932, published Dec. 16, 2021, titled “Miniaturized wearable medication administration device,” this disclosure of which is incorporated herein by reference.

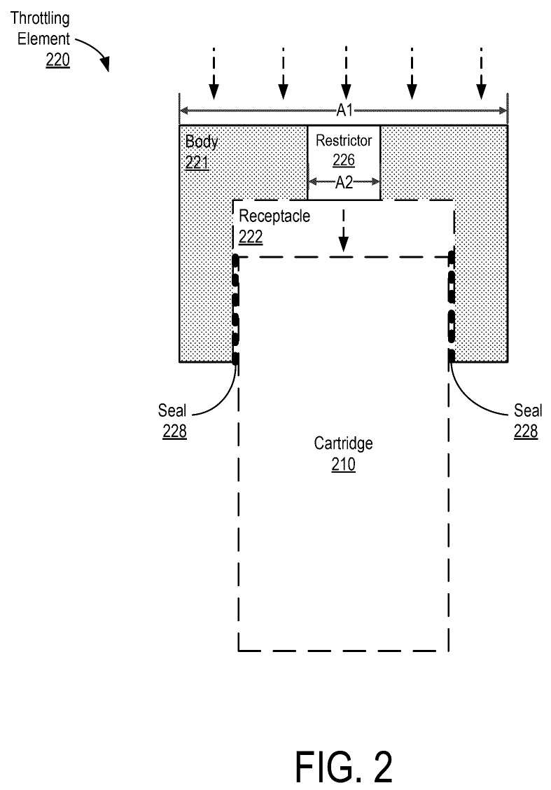

FIG. 2 depicts a more detailed view of a throttling element or assembly 220, according to embodiments. The throttling element 220 can be functionally and/or structurally similar to the throttling element 120. The throttling element 220 can include a body 221 that includes a restrictor 226. Optionally, the body 221 can define a receptacle 222 configured to receive a cartridge 210, which can be functionally and/or structurally similar to the cartridge 110. In particular, the receptacle 222 can be shaped to extend around a bottom heel of the cartridge 210. When the cartridge 210 is received within the receptacle, the body 221 can be configured to fit snugly or closely around the cartridge 210, e.g., such that a seal 228 is formed between the body 221 and the cartridge 210. The seal 228 can be configured to prevent fluid (e.g., intermediary fluid 132) from leaking out around the cartridge 210. As such, any fluid that flows downstream of the restrictor 226 can act on a plunger of the cartridge 210, thereby driving movement of the plunger to deliver a therapeutic agent (e.g., therapeutic agent 114). In some embodiments, the seal 228 is formed by the direct engagement between the body 221 of the throttling element 220 and an outer surface of the cartridge 210. The body 221 of the throttling element 221 can be formed of, for example, an elastomer, which can grip tightly around an outer surface of the cartridge 210. The cartridge 210, similar to the cartridge 110, can be formed of a rigid material, such as, for example, glass.

In some embodiments, the throttling element 220 can be configured to control the rate of needle ejection. In particular, the body 221 of the throttling element 220 can have a proximal end with an area A1. When a stored energy device (e.g., stored energy device 130) drives an intermediary fluid toward the cartridge, the intermediary fluid can act on the area A1 to drive movement of the entire cartridge 210. The cartridge 210, by moving, can then engage with a needle hub of a needle assembly (e.g., needle assembly 140), thereby driving movement of a needle into a patient. The throttling element 220, by being coupled to the proximal end of the cartridge 210, therefore functions as an actuator that drives the ejection of the needle. As such, the rate of needle ejection can be controlled by setting the area A1. In some embodiments, the area A1 can be a circular cross-section having a diameter of between about 20 mm and about 50 mm, including all values and sub-ranges therebetween.

Additionally or alternatively, the throttling element 220 can be configured to control the rate of drug extrusion or delivery. As depicted in FIG. 2, the body 221 of the throttling element 220 can include a restrictor 226. In some embodiments, the restrictor 226 can be an opening or orifice that extends through the body 221 of the throttling element 220. The restrictor 226 can have a cross-sectional area A2 that generates a dynamically choked flow of intermediary fluid. In some embodiments, the cross-sectional area A2 can be a circular cross-section having a diameter of less than about 5 mm, less than about 4 mm, less than about 3 mm, less than about 2 mm, less than about 1 mm, or less than about 0.5 mm, including all values and sub-ranges therebetween (e.g., between about 0.5 mm and about 5 mm, or between about 0.5 mm and about 2 mm). In some embodiments, the cross-sectional area A2 can be less than about 3 mm², and can be circular and/or non-circular. The restrictor 226 can work together with the intermediary fluid to generate the choked flow, which then leads to a constant flow rate through the throttling element 220. The area A2 of the restrictor 226 can be selected to control for pressures generated by the stored energy device that exceed the stalling pressure of the therapeutic agent being delivered.

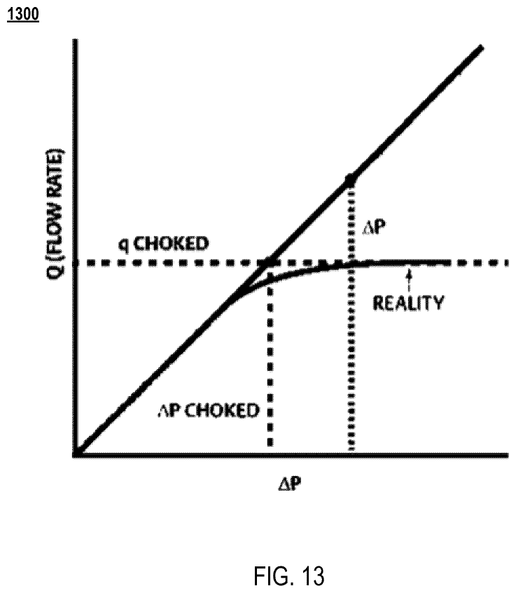

As shown in FIG. 13, the constant flow rate of fluid through a throttling element (e.g., through restrictor 226 of throttling element 220) can be achieved by generating a dynamically choked flow of intermediary fluid. The choked flow of the intermediary fluid is associated with the Venturi effect and is generated when the intermediary fluid passes through a constriction (e.g., restrictor 226) in the throttling element. When the intermediary fluid passes through the constriction into a lower pressure environment, the velocity or flow rate Q of the intermediary fluid increases. At initially subsonic upstream conditions, the conservation of mass principle requires the flow rate Q to increase as the intermediary fluid flows through the smaller cross-sectional area of the constriction. At the same time, the Venturi effect causes the causes the pressure to decrease downstream of the constriction. Choked flow occurs when the mass flow or flow rate Q does not increase with a further decrease in the downstream pressure environment for a fixed upstream pressure and temperature. In other words, beyond a certain difference ΔP between the upstream and downstream pressures, the flow rate Q of the intermediary fluid remains constant (or substantially constant) under adiabatic conditions. As shown in the graph 1300, this occurs beyond ΔP CHOKED.

In the case of a gaseous intermediary fluid, the physical point at which choked flow occurs for adiabatic conditions is when the exit plane velocity is at sonic conditions at or above a Mach number of 1, and is therefore dependent on fluid density. In the case of a liquid intermediary fluid, the physical point at which choked flow occurs for adiabatic conditions is when the pressure drop across the constriction is such that the pressure at the constriction's exit is below the vapor pressure of the intermediary fluid. As such, beyond a certain pressure difference ΔP CHOKED, the mass flow rate through the throttling element cannot be exceeded, which thereby limits the pressure action on the plunger of the cartridge and the drug extrusion rate.

In some embodiments, variable density intermediary fluids or other fluid modifications can also be used to further adjust or control the flow rate of the intermediary fluid and, consequently, the rate of drug extrusion. In some embodiments, the intermediary fluid can be a Newtonian fluid, such as, for example, water, oil, alcohol, or combination thereof. In some embodiments, the intermediary fluid can be a non-Newtonian fluid, such as a slurry, a gel, a colloid, etc. Depending on the flow rate desired (e.g., due to the needle gauge, properties of the therapeutic agent, etc.), the throttling element 220 can be selected to operate with a Newtonian or non-Newtonian fluid.

In therapeutic delivery systems, it can be desirable to use a single stored energy source for both needle ejection and drug extrusion. This can reduce the manufacturing costs as well as allow for smaller profile devices (e.g., with less components). With a single stored energy source and no throttling element, the same pressure would be applied for needle ejection and drug extrusion. This can lead to complications, especially with non-Newtonian fluids being delivered and small needle gauges. In particular, it can be desirable to have needles with small profiles to reduce patient discomfort. But small needles can result in greater pressures, especially with non-Newtonian fluids, and therefore cause cartridge failure (e.g., breaking of the cartridge body) and stalling of therapy delivery. The risk of cartridge failure also increases with larger size cartridges, which may be needed for delivering larger doses of a therapeutic agent. With a throttling element, these issues can be avoided as different pressures can be applied for needle ejection and drug extrusion, e.g., due to the different sizes of areas A1 and A2, as described above with reference to FIG. 2.

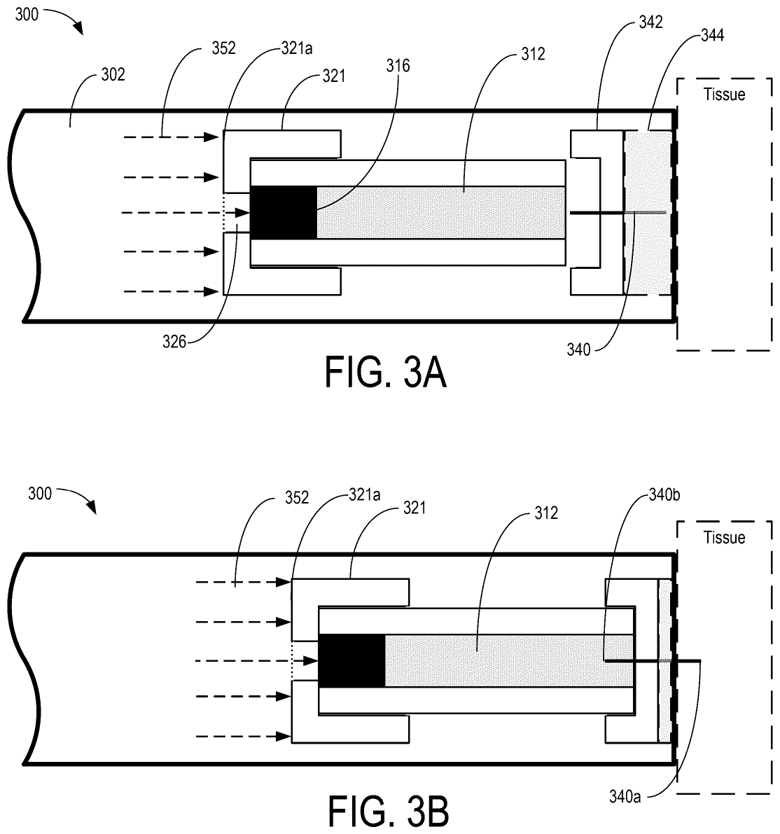

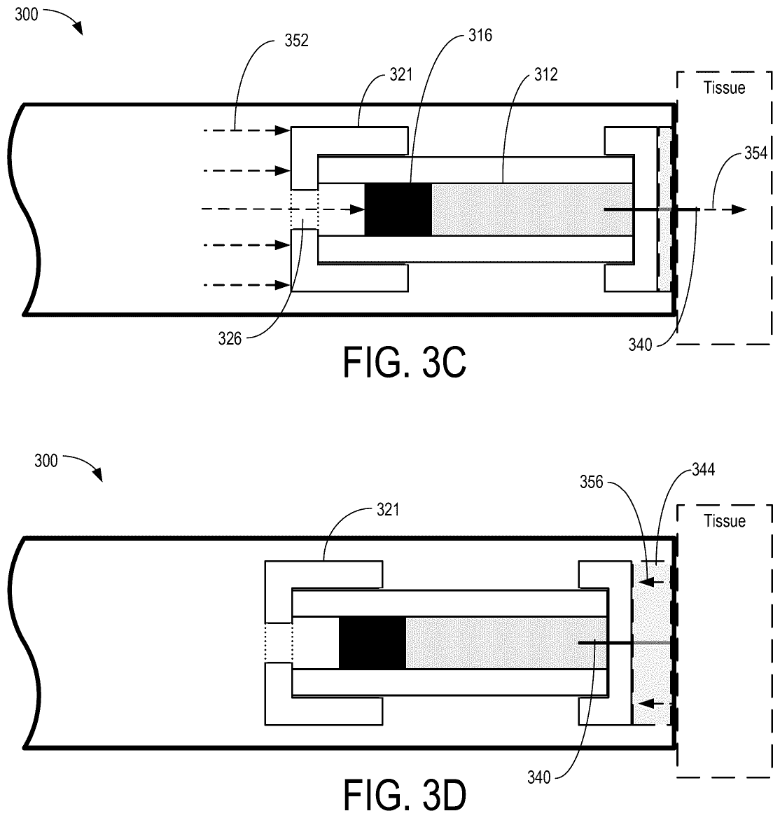

FIGS. 3A-3D schematically depict the operation of a therapeutic delivery system at different stages, according to embodiments. FIG. 3A depicts a therapeutic delivery system 300 when the system 300 has been placed against a tissue surface of a patient. The therapeutic delivery system 300 can be structurally and/or functionally similar to other therapeutic delivery systems described herein, including, for example, therapeutic delivery system 100. For example, the therapeutic delivery system 300 can include a housing 302 that contains or houses a throttling element with a body 321, a cartridge having a plunger 321 and a reservoir 312, and a needle assembly having a needle 340 and a needle hub 342. While not depicted, the therapeutic delivery system 300 can also include a stored energy device (e.g., stored energy device 130).

As depicted in FIG. 3A, the stored energy device has been activated, and a flow 352 of intermediate fluid (e.g., a pressurized or hydraulic fluid) is acting on a proximal side 321a of the body 321 of the throttling element. The throttling element body 321 is coupled to a proximal end (e.g., a bottom heel) of the cartridge. When the flow 352 of the intermediate fluid acts on the proximal side 321a of the throttling element body 321, this causes the throttling element and the cartridge to move toward the needle 340.

The needle 340 and the needle hub 342 may be held in position within the housing 302 by a support 344. The support 344 can be, for example, an elastic or a deformable element. In an embodiment, the support 344 can be a spring. The needle 340 can be a double-sided needle, e.g., having a distal end 340a and a proximal end 340b that are sharp and can puncture through tissue or other surfaces. When the flow 352 of the intermediate fluid acts on the throttling element and the cartridge, this drives the distal end of the cartridge into the needle hub 342, which then pushes against the support 344. The stored energy device can be configured to generate the flow 352 such that sufficient force is applied on the cartridge and therefore on the needle hub 352 to overcome the force of the support 344 that holds the needle 340 and needle hub 342 in place. Once this force of the support 344 is overcome, the distal end 340a of the needle 340 can extend out of the housing 302 and into the tissue, as depicted in FIG. 3B. At or around the same time, the proximal end 340b of the needle 340 can puncture through a distal seal of the cartridge and into the reservoir 312.

Once the needle 340 has extended into the tissue and punctured into the reservoir 312, the needle 340 can act as a fluid passageway for delivering a therapeutic agent disposed within the reservoir 312 into the tissue. As depicted in FIG. 3C, the flow 352 of the intermediary fluid can continue to act on the throttling element. The throttling element can have a restrictor 326 that allows a reduced flow of the intermediary fluid to flow downstream of the throttling element. In embodiments, the restrictor 326 can have a cross-sectional area that is sufficiently small for generating a choked flow of intermediary fluid downstream of the restrictor 326. As described with reference to FIG. 13, the choked flow of the intermediary fluid can be associated with a constant flow rate, which then acts on the plunger 316 of the cartridge, moving the plunger distally (e.g., toward the needle 340). The distal movement of the plunger 316 then causes an amount or volume 354 of the therapeutic agent contained within the reservoir 312 to exit the needle 340 into the tissue.

Once the dose of the therapeutic agent has been delivered, the stored energy device can stop generating the flow 342 of the intermediary fluid. In FIG. 3D, the stored energy device has stopped generating the flow 352 of the intermediary fluid, e.g., due to the stored energy dissipating or the stored energy device deactivating (e.g., turning off). When this flow stops, the support 344 can then revert 356 to its original configuration, thereby retracting the needle 340 back into the housing 302. The therapeutic delivery system 300 can then be removed from the tissue.

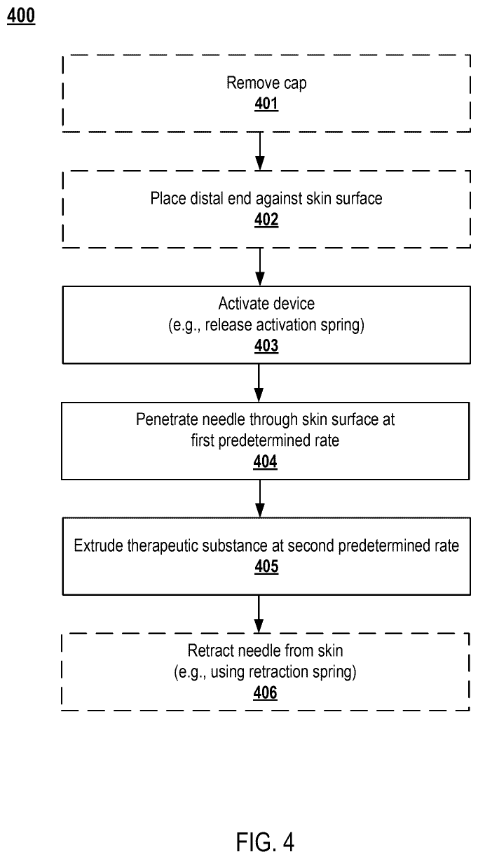

FIG. 4 is a flow chart of an example method 400 of using a therapeutic delivery system as described herein (e.g., therapeutic delivery system 100, 300, etc.), according to embodiments. The method 400 can optionally include removing a cap, at 401. For example, in some embodiments, the therapeutic delivery system can include a main body or housing and a removable cap. The cap can be placed over the main body such that a needle ejection port and/or an activation device of the therapeutic delivery system are covered. As such, prior to use, the cap can be removed.

Optionally, at 402, a distal or dispensing end of the therapeutic delivery system can be placed against a skin surface of the patient. The dispensing end of the therapeutic delivery system can include a surface for resting against the skin surface. In some embodiments, the surface of the dispensing end of the therapeutic delivery system can have a shape designed for mating against or engaging with the skin surface, such as, for example, a curved or concave shape.

At 403, the therapeutic delivery system can be activated, e.g., by actuating an activation device. As described with reference to FIG. 1, the therapeutic delivery system can include an activation device, such as, for example, a button, switch, etc., which can be actuated to activate a stored energy device (e.g., stored energy device 130) of the therapeutic delivery system. For example, an activation button can be pushed to activate the stored energy device. In some embodiments, the stored energy device can include a spring that, upon activation, releases from a compressed, high-energy state to generate a force that drives the flow of an intermediary fluid. Alternatively, the stored energy device can include an electric motor or other mechanism for driving the flow of an intermediary fluid.

In response to the activation of the device, and consequently the generation of the flow of intermediary fluid, a needle of the therapeutic delivery system can be inserted into the patient through the skin surface, at 404. As described with reference to FIGS. 3A and 3B, the flow of intermediary fluid can act on a throttling element and a cartridge, which move distally to eject the needle from a housing of the therapeutic delivery system. The needle can be ejected at a first predetermined rate, e.g., as controlled by the size or area of the proximal side of the throttling element.

At 405, a therapeutic substance or agent can be extruded from the cartridge of the therapeutic delivery system and into the patient. The therapeutic substance extrusion can occur at a second predetermined rate that is different from the first predetermined rate associated with the needle ejection. For example, the second predetermined rate can be less than the first predetermined rate. In particular, the therapeutic substance extrusion can be driven by the movement of a plunger. The plunger can move in response to a flow of the intermediary fluid acting on the proximal side of the plunger. The plunger can be disposed downstream of a throttling element (e.g., throttling element 320), which can be configured to reduce the flow of the intermediary fluid that acts on the plunger. In some embodiments, the throttling element can include a restriction that is configured to generate a choked flow of the intermediary fluid. The choked flow can be associated with a constant flow rate, which exerts less pressure or force on the plunger than the full force of the intermediary fluid. The choked flow at this constant flow rate then acts on the plunger to drive the extrusion of the therapeutic substance at a second predetermined rate that is less than the first predetermined rate of needle ejection.

After the appropriate dose of the therapeutic substance has been delivered to the patient, the needle optionally can be retracted from the skin, at 406. In some embodiments, the therapeutic delivery system can include a retraction spring that is coupled to the needle hub, and once the flow of intermediary fluid halts, this retraction spring can be configured to pull or push the needle (e.g., depending on spring configuration) back into the housing of the therapeutic delivery system. In such embodiments, the power spring compresses the retraction spring during delivery of the drug, and once the pressure to the intermediary fluid is removed, the retraction spring is able to retract the needle.

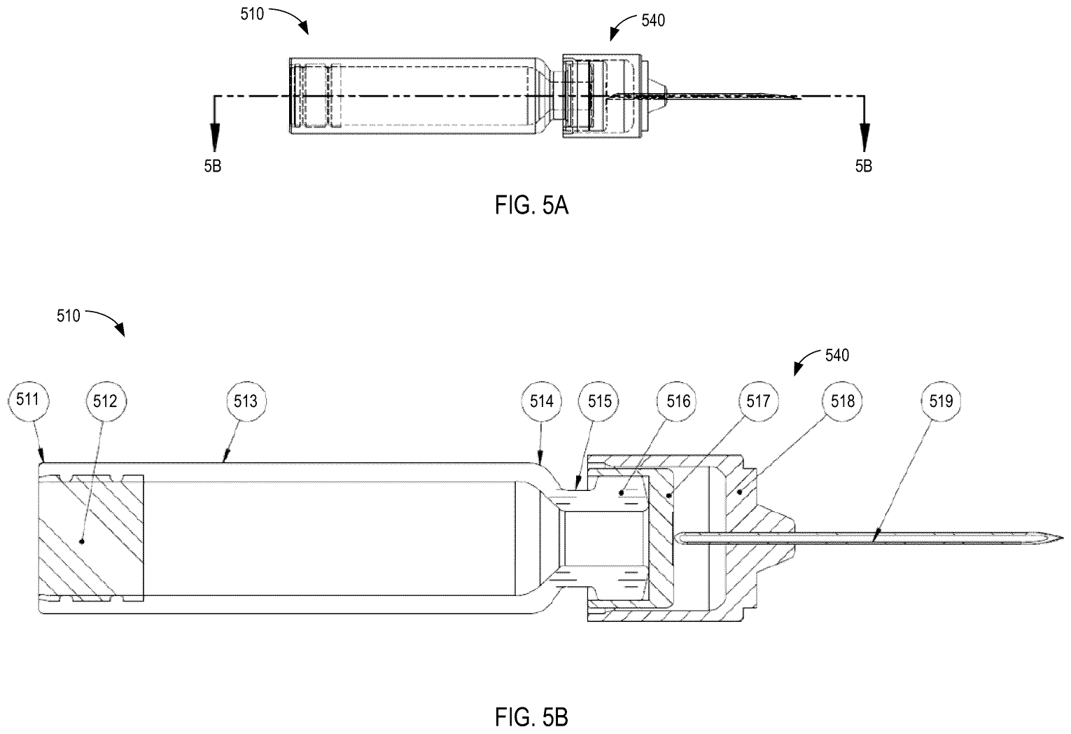

FIGS. 5A and 5B depict views of a cartridge 510 and a needle assembly 540 of a therapeutic delivery system, according to embodiments. FIG. 5A depicts a side view of the cartridge 510 and the needle assembly 540, while FIG. 5B depicts a cross-sectional view of the cartridge 510 and the needle assembly 540 along line 5B-5B shown in FIG. 5A. The cartridge 510 and the needle assembly 540 can be structurally and/or functionally similar to other cartridges and needle assemblies described herein, including, for example, cartridge 110 and needle assembly 140. The cartridge 510 can include a glazed end of heel 511, a plunger 512, a body implemented as a barrel 513 with an elongate region that extends to a shoulder region 514 and a neck region 515, a crown or distal end 516, and a septum or seal implemented as a crimp cap 517. The needle assembly 540 includes a needle 519 and a needle hub 518. In some embodiments, the cartridge 510 can be a glass cartridge or container.

The barrel 513 can include an inner surface, an outer surface, and a wall thickness extending between the inner and outer surfaces. In some embodiments, the wall of the barrel 513 can be formed of glass, such as, for example, borosilicate glass. The barrel 513, together with the plunger 512 and the crimp cap 517 can define a reservoir for containing a therapeutic agent. The therapeutic agent can be extruded from the reservoir via the needle 519. The crimp cap 517 can be a crimp cap combi-seal, although in other embodiments, other types of seals can be used. The distal end 516 of the cartridge 510 can be configured to receive the needle hub 518 thereon. The needle hub 518 can be coupled to the distal end 516 of the cartridge 510 by any known technique, such as a threaded connection or snap fit configuration. The needle hub 518 can support the needle 519. The needle 519 can be a double-ended needle with a proximal end that can pierce through the crimp cap 517 and a distal end that can puncture into tissue. In use, the needle 519 can move relative to the cartridge 510 such that the proximal end of the needle 519 pierces through the crimp cap 517 and gains access to the reservoir containing the therapeutic agent. The distal end of the needle 519 can also puncture into tissue, thereby establishing a fluid pathway between the reservoir and the tissue for delivery of the therapeutic agent.

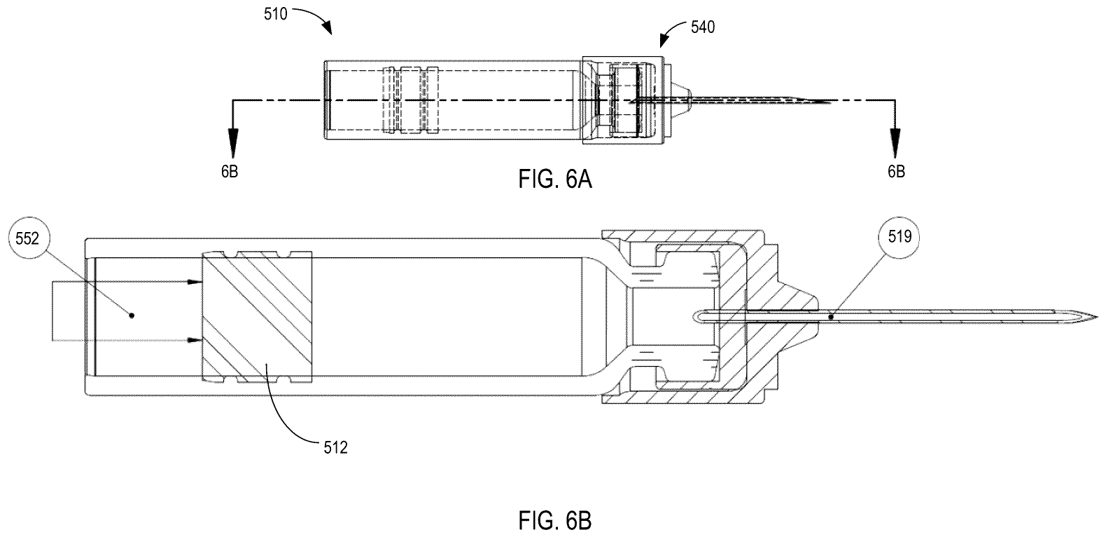

The plunger 512 of the cartridge 510 can be configured to advance distally to extrude the therapeutic agent contained within the reservoir. The plunger 512 can advance distally in response to a pressure applied by a stored energy device (not depicted). FIG. 6B schematically depicts this pressure 552 acting on the plunger. In some embodiments, the stored energy device can include a spring that drives an intermediary fluid, which applies pressure to the proximal side of the plunger 512 to advance the plunger 512 distally. FIG. 5B depicts the plunger 512 in its initial position. FIG. 6B depicts the plunger 512 after it has advanced distally in response to the pressure 552.

In some embodiments, the proximal end or heel 511 of the cartridge 510 can be coupled to (e.g., fitted with) a fluidic throttling element (e.g., throttling element 120), e.g., for facilitating delivery of constant pressure to the cartridge 510 and for controlling a drug extrusion rate. The throttling element can be configured to provide drug extrusion with constant pressure despite variable or decreasing force applied by the stored energy device. The throttling element can be configured to deliver a throttled and constant (or substantially constant) flow of an intermediary fluid to the plunger 512, e.g., thereby driving a constant rate of advancement of the plunger 512 and therefore a constant rate of therapeutic agent delivery or extrusion. In embodiments, the throttling element includes an opening or orifice that replaces the needle 519 as the fluid rate limiting orifice, thus modulating the flow rate of drug extrusion.

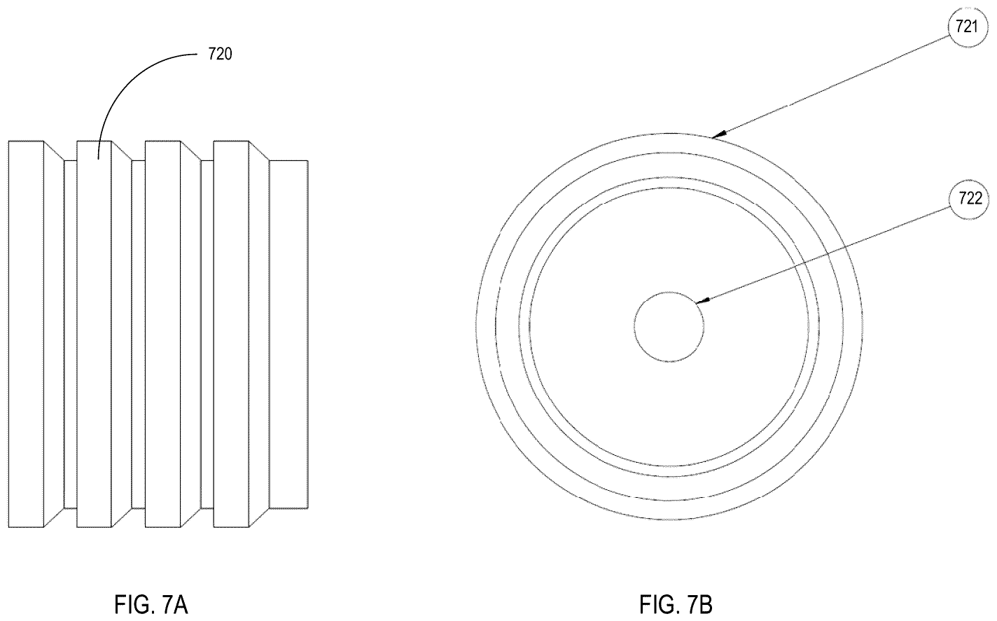

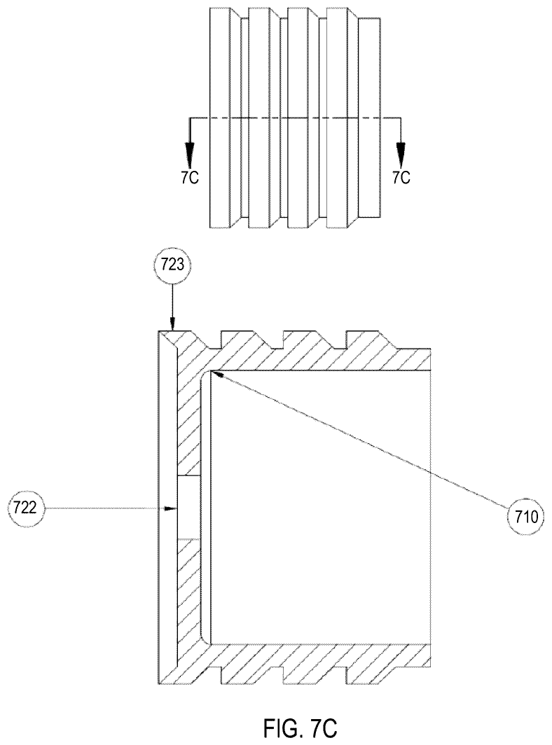

FIGS. 7A-7C depict different views of a throttling element 720, according to embodiments. The throttling element 720 can be structurally and/or functionally similar to other throttling elements described herein, including, for example, throttling elements 120 and 220. FIG. 7A depicts a side view of the throttling element 720, FIG. 7B depicts a front view of the throttling element 720, and FIG. 7C depicts a cross-sectional view of the throttling element. As depicted in these figures, the throttling element 720 can be formed as a monolithic structure. Alternatively, the throttling element 720 can be formed of a plurality of sub-parts or components that are joined together. The throttling element 720 can include a body 721 that defines an outer seal 723 and an orifice 722. The outer seal 723 can be configured to seal against an inner surface of a housing (e.g., housing 102) of a therapeutic delivery system, thereby preventing leakage of an intermediary fluid around the outside of the throttling element 720. The orifice 722 can be an example of a restrictor (e.g., restrictor 226). In embodiments, the orifice 722 can be configured to act as the rate limiting point in a therapeutic delivery system including the throttling element 720. For example, the orifice 722 can be configured to have a cross-sectional area that is sufficiently small to induce a throttled flow of intermediary fluid. As described with reference to FIG. 13, such a throttled flow can be associated with a constant flow rate (or substantially constant flow rate), and therefore enable a more constant drug extrusion rate.

The throttling element 720 can be fitted to the back or heel of a cartridge. FIG. 7C depicts the interface 710 between the heel of the cartridge and the throttling element 720. In embodiments, the throttling element 720 can seal around the heel of the cartridge. In certain examples, the throttle can be formed from one or more medical grade elastomers, such as, for example, liquid silicone rubber (LSR), fluoroelastomers (FKM), perfluoroelastomers (FFKM), and ethylene-propylene diene monomer (EPDM). In embodiments, the throttling element 720 can have a minimum Shore hardness of 40A.

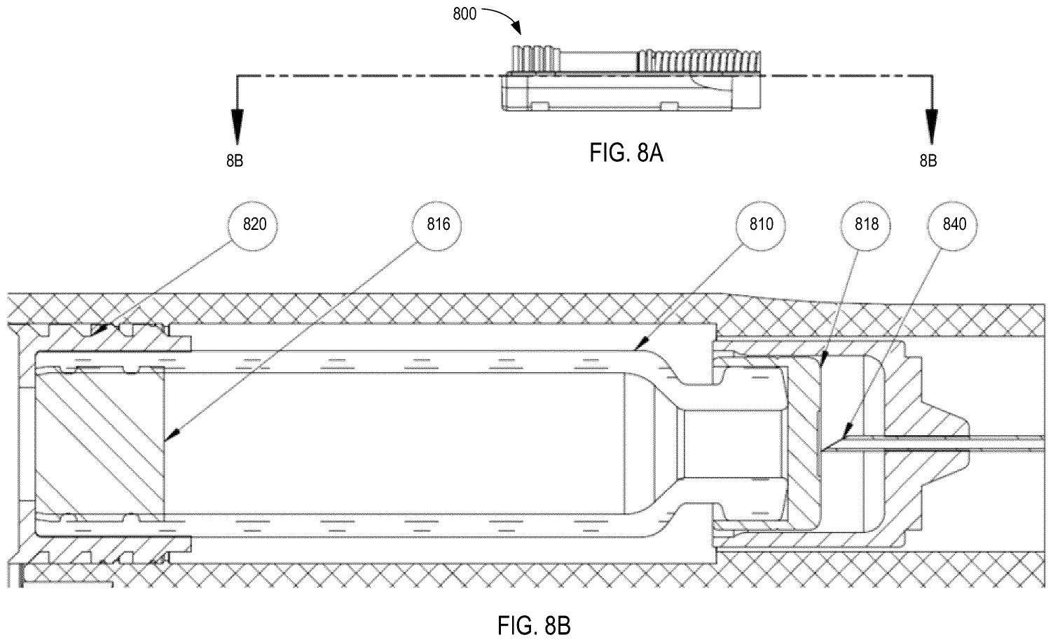

FIGS. 8A and 8B depict views of a therapeutic delivery system 800, including a cartridge 810 that is fitted with a throttling element 820, according to embodiments. FIG. 8A depicts a side view of the system 800, and FIG. 8B depicts a partial cross-sectional view of the system 800, taken along line 8B-8B depicted in FIG. 8A. The therapeutic delivery system 800 can be structurally and/or functionally similar to other therapeutic delivery systems described herein, including, for example, therapeutic delivery system 100. For example, the therapeutic delivery system 100 can be a needle-based delivery system and can include a needle 840. The cartridge 810 can include a plunger 816 and a seal implemented as a crimp cap 818. The cartridge 810 and the needle 840 can be structurally and/or functionally similar to the cartridge 510 and the needle 519 described with reference to FIGS. 5A-6B (as well as other cartridges and needles described herein), and therefore are not described in detail again herein.

As depicted in FIG. 8B, the throttling element 820 can extend over the bottom heel of the cartridge 810 to restrict at least a portion of the flow of the intermediary fluid to the plunger 816. The throttling element 820 can be structurally and/or functionally similar to other throttling elements described herein, including, for example, throttling element 120, 220, or 720. The throttling element 820 can include an orifice that can replace the orifice of the needle 840 as the rate limiting orifice in the system 800. By replacing the needle 840 as the rate limiting orifice, the throttle can prevent and/or mitigate the risk of blockage in the needle 840 and/or breakage of the cartridge 810 during delivery of a therapeutic agent (e.g., a standard, viscous, shear sensitive, or non-Newtonian therapy).

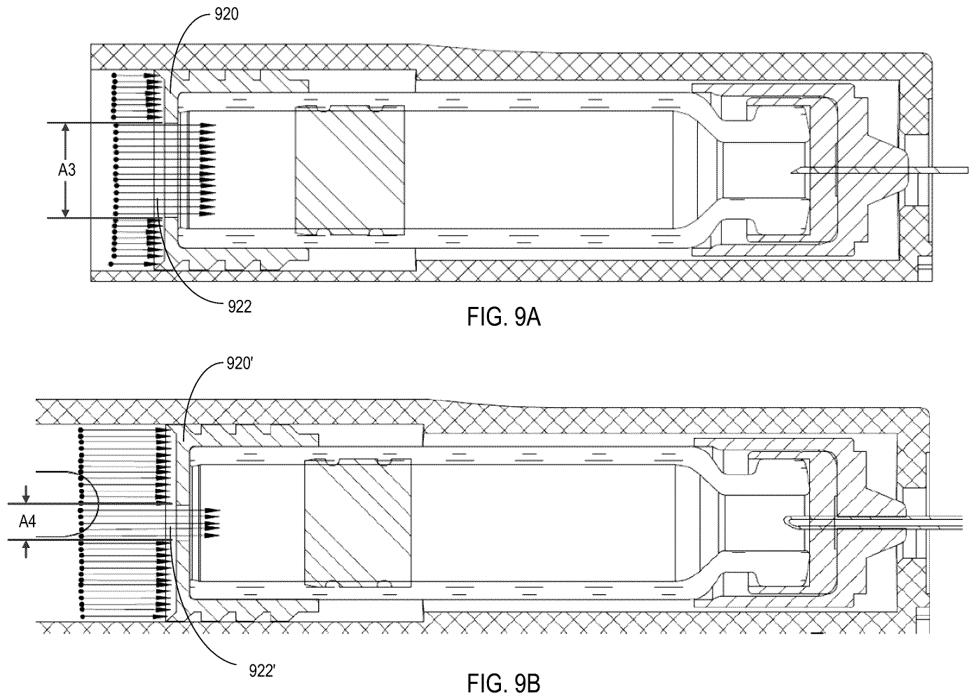

The size of the restrictive orifice of a throttling element can be varied, e.g., to increase or decrease the rate of drug extrusion given needle gauge, therapeutic substance sensitivities or properties, etc. FIGS. 9A and 9B depict throttling element 920, 920′ with different size restrictive openings 922, 922′. As shown restrictive opening 922 of throttling element 920 can have an area A3, while restrictive opening 922′ of throttling element 920′ can have an area A4 which is smaller than the area A3. The area of the throttling element can be reduced from A3 to A4 to reduce the rate of drug extrusion, e.g., to avoid blockage within a needle. As described above with reference to FIG. 13, the throttling elements 920, 920′ can be configured to provide constant flow of intermediary fluid therethrough, and therefore provide for constant rates of drug or other therapy extrusion. Therefore, keeping other operational parameters equal (e.g., the operating forces of the stored energy device, the type of intermediary fluid, etc.), the rate of drug extrusion can be controlled by selecting throttling elements with different sized orifices.

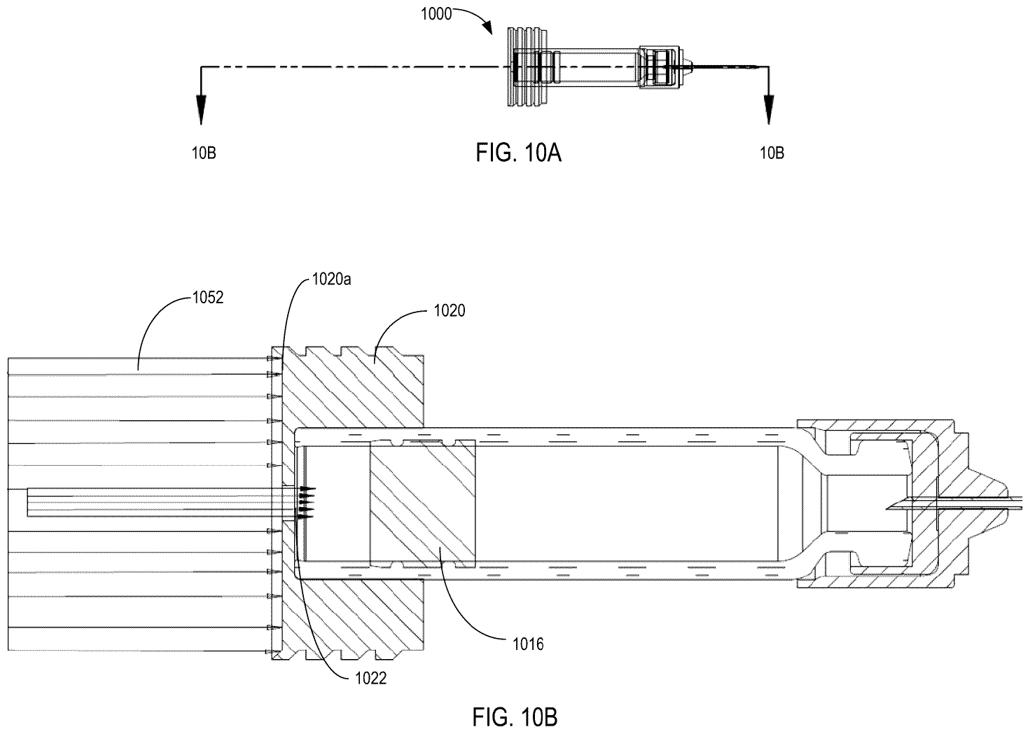

In some embodiments, a throttling element can also be used to control the rate of needle insertion. In particular, the outer diameter of the proximal end of a throttling element can be varied to control the initial rate that the needle is ejected from a therapeutic delivery system and into a patient. FIGS. 10A and 10B depict a therapeutic delivery system 1000, where a throttling element 1020 is configured to control the rate of needle ejection, according to embodiments. The therapeutic delivery system 1000 can be structurally and/or functionally similar to other therapeutic delivery systems described herein, including, for example, therapeutic delivery systems 100, 800, etc. For example, the therapeutic delivery system can include a cartridge with a plunger 1016 and a needle assembly. The throttling element 1020 can be fitted over the heel of the cartridge, similar to other throttling elements 1020 described herein. The throttling element 020 can include an opening or orifice 1022, which can have a smaller area that restricts the flow 1052 of an intermediary fluid to thereby reduce the rate of drug extrusion. Additionally, the throttling element 1020 can include a proximal surface 1020a having a larger area upon which the flow 1052 of the intermediary fluid can act to cause ejection of the needle. As described with respect to FIGS. 3A-3D, when the flow 1052 of intermediary fluid acts upon the proximal surface 1020a of the throttling element 1020, this can cause the cartridge to advance distally to engage with a needle hub. Such advancement can cause a proximal end of the needle to pierce through a seal or septum of the cartridge, while also advancing the needle out of a housing of the therapeutic delivery system. The throttling element 1020 therefore can control the needle ejection rate, and in particular, the area of the proximal side 1020a of the throttling element 1020 determines the initial force that the needle is ejected from the therapeutic delivery system.

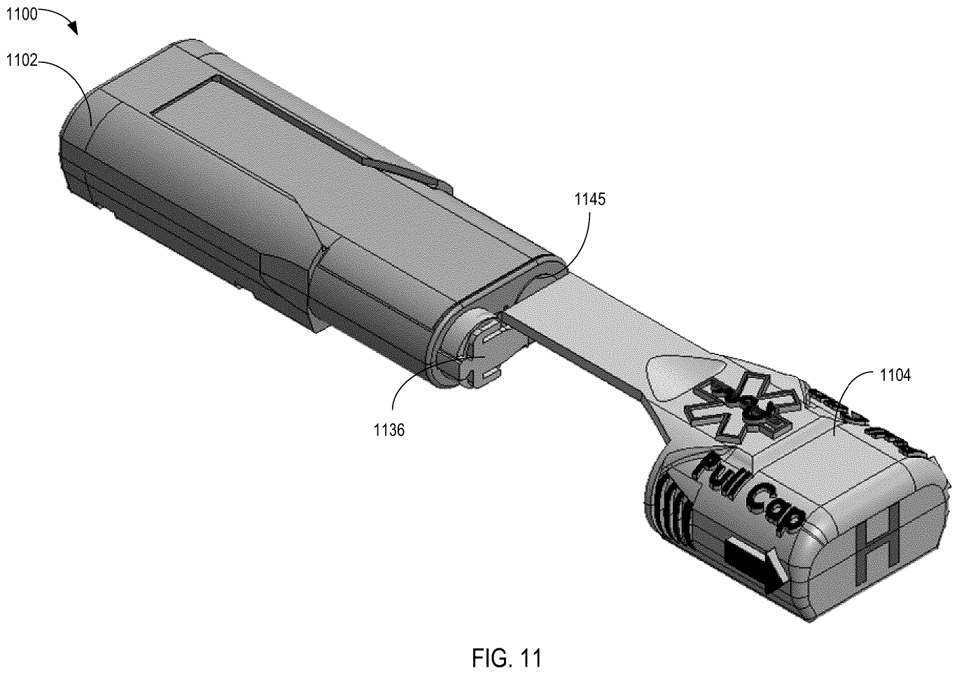

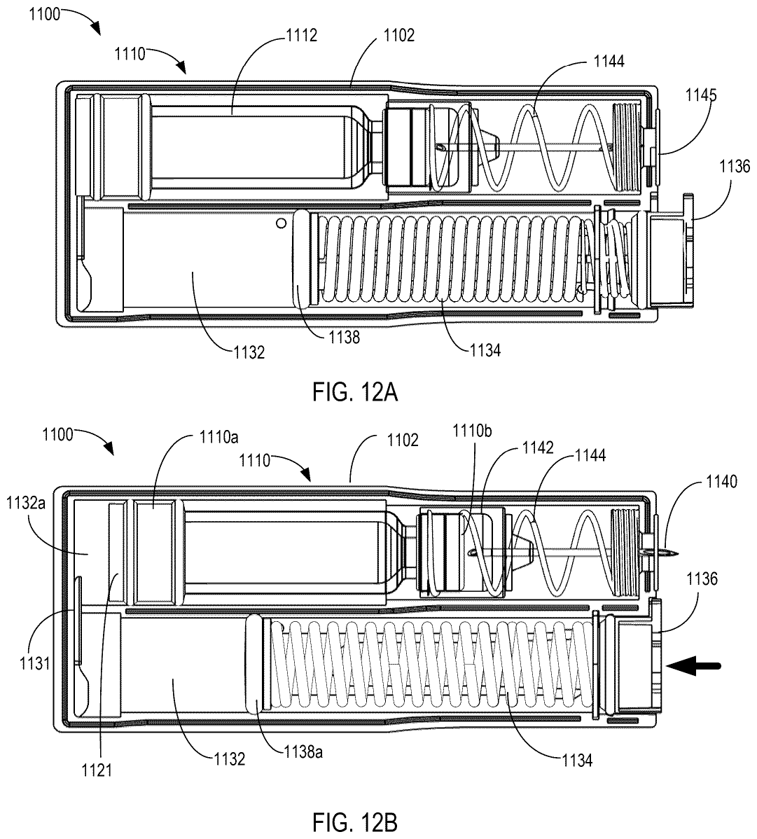

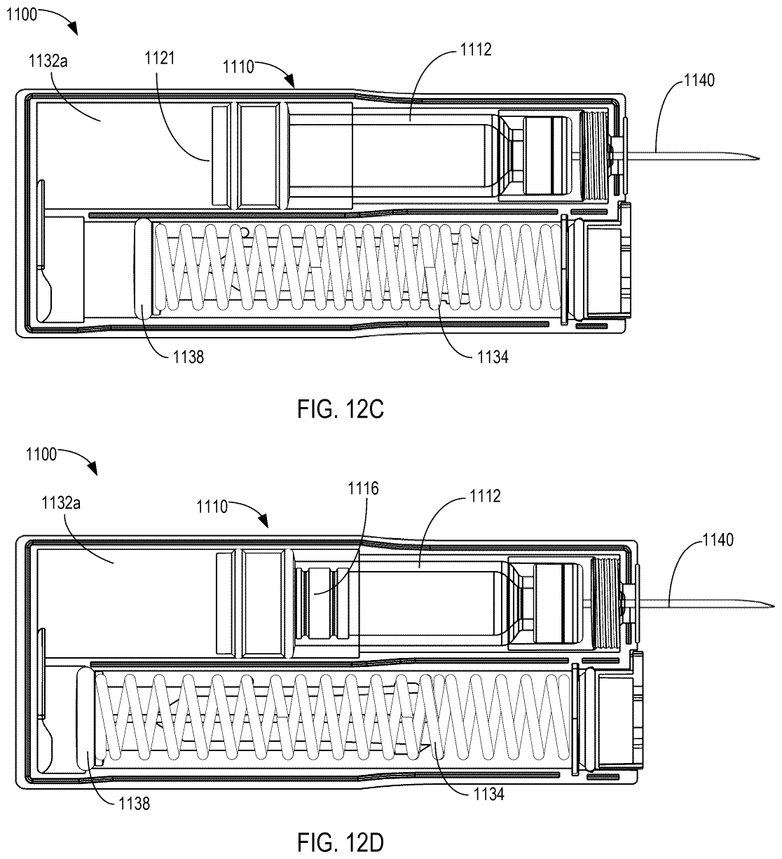

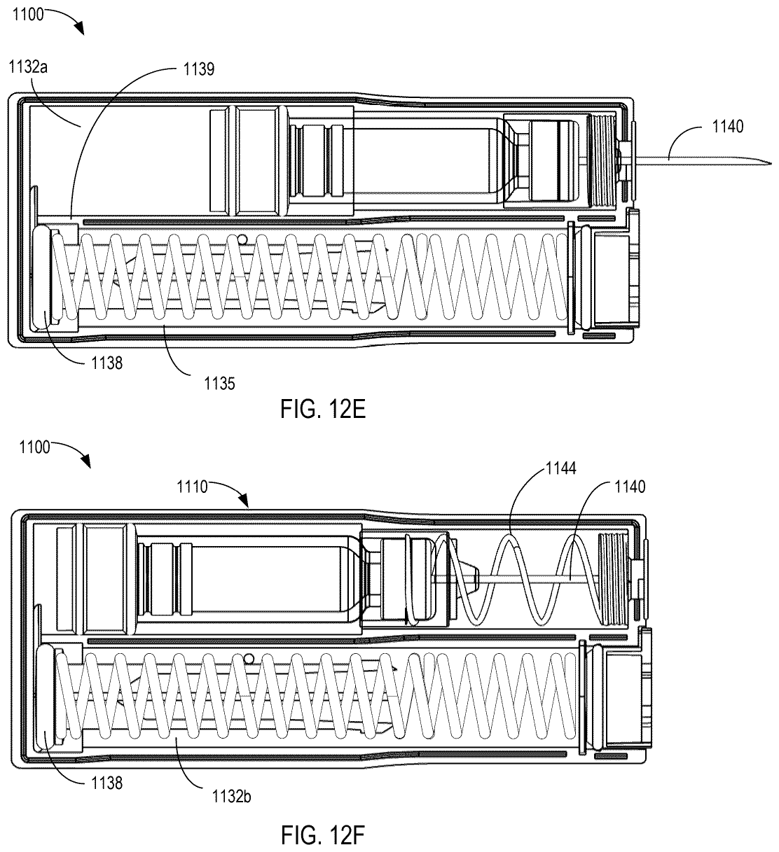

Referring now to FIGS. 11-12F, another example of a therapeutic delivery system 1100 is described. FIG. 11 depicts a schematic view of the therapeutic delivery system 1100, while FIGS. 12A-12F depict the therapeutic delivery system 1100 at different stages during the operation of the system. The therapeutic delivery system 1100 can be structurally and/or functionally similar to other therapeutic delivery systems described herein, including, for example, therapeutic delivery systems 100, 800, 1000, etc. The therapeutic delivery system 1100 can be a needle-based injection system.

As depicted in FIG. 11, the system 1100 can include a main body or housing 1102 and a cap 1104. The housing 1102 can contain other components of the system 1100, including, for example, a cartridge 1112, a stored energy device, a needle assembly including a hub 1142 and needle 1140, etc., as depicted in FIGS. 12A-12F. The cap 1104 can be configured to cover a portion of the therapeutic delivery system 1100. In FIG. 11, the cap 1104 is shown removed from the housing 1102, e.g., to expose one or more other components of the therapeutic delivery system 1100. In particular, the removal of the cap 1104 exposes an activation device 1136 of the therapeutic delivery system 1100. The activation device 1136 can be configured to activate a stored energy device that includes a spring 1134 (e.g., a deployment spring) and a piston 1138. The removal of the cap 1104 also exposes a septum 1145 that covers an opening through which a needle 1140 can be ejected out of the housing 1102.

Prior to use, the cap 1104 can be removed from the housing 1102 to expose the activation device 1136 and the needle ejection point. The housing 1102 can then be pressed against an injection site (e.g., against skin or against clothing overlaying the injection site) so as to position the needle for insertion into the injection site. FIG. 12A depicts the system 1100 with the cap 1104 removed and ready for activation. At this stage, the spring 1134 of the stored energy device is loaded in a high-energy state, e.g., a fully compressed state, and the piston 1138 of the stored energy device is in a first position, e.g., a fully retracted position. The piston 1138 can include a seal 1138a at its distal end that is configured to act on an intermediary fluid 1132 disposed within the housing 1102 of the system 1100.

The activation device 1136 can be a button or trigger. The activation device 1136, when pressed or actuated as shown in FIG. 12B, can release the stored energy device, e.g., by releasing the spring 1134 and/or piston 1138. The spring 1134 can then apply a force that drives the movement of the piston 1138, which moves relative to the housing 1102 and applies pressure to the intermediary fluid 1132. The intermediary fluid 1132, when pressurized, can be driven through a passageway including a channel 1131 into a region that is adjacent to a throttling element 1121. The throttling element 1121 can be fitted over a proximal end or heel 1110a of the cartridge. In some embodiments, the actuation of the activation device 1136 can release a locking mechanism (e.g., a split lock, a pin, etc.) that allows the piston 1138 to move along the housing 1102. In some embodiments, once the piston 1138 has been unlocked, the spring 1134 drives the piston 1138 along a first portion of the housing 1102 in a direction away from the activation device 1136. The piston 1138, when driven, can act on the intermediary fluid 1132, which then acts on the cartridge 1112 and the needle 1140. In particular, the movement of the piston 1138 can generate a flow 1132a of intermediary fluid 1132 that acts on the proximal side of the throttling element 1121. The flow 1132a of the intermediary fluid 1132 acting on the throttling element 1121 can cause the cartridge 1110, the needle hub 1142, and the needle 1140 to move toward the septum 1145. The needle 1140 can be a double-ended needle, i.e., a needle that has a sharp proximal end and a sharp distal end. The distal end of the needle 1140 can be configured to pierce through the septum 1145, while the proximal end of the needle 1140 can be configured to pierce through a seal or septum of the cartridge 1110.

In some embodiments, the needle hub 1142 and the needle 1140 can be spaced from the septum 1145 (and therefore held within the housing 1102) by a compression spring 1144 (e.g., a retraction spring). Therefore, to be able to eject the needle 1140 from the housing 1102, the spring 1134 of the stored energy device must be configured to generate sufficient energy via the flow 1132a of the intermediary fluid 1132 to overcome the force of the spring 1144. In some embodiments, the spring 1134 can be a compression spring with a spring rate of between about 1 N/mm and about 3 N/mm, including all values and sub-ranges therebetween, including, for example, about 1.50 N/mm, about 1.55 N/mm, about 1.60 N/mm, about 1.65 N/mm, about 1.70 N.mm, about 1.75 N/mm, about 1.80 N/mm, about 1.85 N/mm, about 1.90 N/mm, about 1.95 N/mm, or about 2.00 N/mm. In some embodiments, the spring 1144 can be a compression spring with a spring rate that is less than the spring rate of the spring 1134. In some embodiments, the spring 1144 can be a compression spring with a spring rate of between about 0.5 N/mm and about 2 N/mm, including all values and sub-ranges therebetween, including, for example, about 1.0 N/mm, about 1.1 N/mm, etc. In some embodiments, the spring 1134 and/or the spring 1144 can be formed of a metal or metal composite. In some embodiments, the spring 1134 can have an outside diameter of between about 5 mm and about 15 mm, including all values and sub-ranges therebetween, including, for example, between about 8.5 mm and about 9.5 mm. In some embodiments, the spring 1144 can have an outside diameter that is the same or substantially the same as the spring 1134, while in other embodiments, the spring 1144 can have an outside diameter that is less than or greater than the spring 1134.

The piston 1138 can continue to move, driving the movement of the cartridge 1110 and therefore the needle hub 1142 and the needle 1140 until the needle hub 1142 engages with a portion of the housing 1102 that prevents further movement of the needle hub 1142 and/or the spring 1144 is fully compressed. At this point, the sharp distal end of the needle 1140 has pierced through the septum 1145 and can be in a fully extended position. FIG. 12C depicts the needle 1140 in its fully extended position. As shown in FIG. 12C, the piston 1138 has advanced further downstream from the activation device 1136. While the cartridge 1110, needle hub 1142, and needle 1140 are driven by the intermediary fluid, a proximal end of the needle 1140 can also pierce through a seal or septum disposed at the distal end 1110a of the cartridge 1110, thereby gaining access to a therapeutic agent contained within a reservoir 1112 of the cartridge 1110. The needle 1140 can then act as a fluid passageway for communicating the therapeutic agent within the reservoir 1112 to the patient.

The throttling element 1121, similar to other throttling elements described herein, can be configured to determine the rate of needle ejection. In particular, the area of the proximal side of the throttling element 1121 upon which the intermediary fluid acts can be sized to produce a predetermined force for driving the movement of the cartridge 1110 and the needle 1140. This area can be increased to increase the force and therefore the ejection rate of the needle 1140 or decreased to decrease the force and therefore the ejection rate of the needle 1140. The throttling element 1121, similar to other throttling elements described herein, can also include a restrictor such as, for example, an opening or orifice. The restrictor can be configured to reduce the flow of the intermediary fluid that acts on the plunger 1116 of the cartridge. More specifically, the restrictor can be configured to generate a throttled or constant flow of the intermediary fluid. As described above, this throttled flow of the intermediary fluid can drive the therapeutic extrusion rate while avoiding failure of the delivery system, e.g., by avoiding high pressures from being generated in the cartridge 1110 and/or blockage in the needle 1140.

As the piston 1138 moves to the position shown in FIG. 12D, the restricted flow of the intermediary fluid can cause the plunger 1116 of the cartridge 1112 to move toward the needle 1140, thereby extruding an amount or volume of the therapeutic agent contained within the cartridge 1112. In some embodiments, continued motion of the piston 1138, e.g., to the position shown in FIG. 12E, can cause the head of the piston 1138 to pass a pressure relief point 1139. In some embodiments, the pressure relief point 1139 can be an opening or bore within the housing 1102 that releases the seal that the piston 1138 has against the inner walls of the housing 1102, thereby releasing the pressure exerted by the piston 1138 on the intermediary fluid while at least a portion 1132b of the intermediary fluid 1132 flows into a relief area within the housing 1102. For example, a portion 1132b of the intermediary fluid 1132 can flow past the head of the piston 1138, e.g., into a region adjacent to the activation device 1136. This can cause a release or decrease in pressure upstream of the throttling element 1121, thereby stopping or reducing further downstream flow of the intermediary fluid 1132 and further movement of the plunger 1116. In some embodiments, the pressure relief point 1139 can be an opening or bore in the wall of the housing 1102, which allows the intermediary fluid 1132 to flow into a relief area (e.g., an area upstream of the piston 1138). This can similarly cause the flow of the intermediary fluid 1132 to stop acting on the plunger 1116. The piston 1138 can continue to drive the movement of the plunger 1116 (and therefore the extrusion of the therapeutic agent) until the piston 1138 has passed the pressure relief point 1139. The location of the pressure relief point 1139 can be selected to control an amount or volume of therapeutic agent that is extruded through the needle 1140. In some embodiments, the dosage amount is between about 100 μL and about 1 mL, including all values and sub-ranges therebetween.

Once pressure applied by the intermediary fluid has abated, the spring 1144 can force the needle hub 1142, the cartridge 1110, and the needle 1140 away from the septum 1145, thereby retracting the needle 1140 to a position that is entirely within the housing 1102. In some embodiments, the spring 1144 is selected so as to be strong enough to provide an immediate or near-immediate retraction of the needle 1140 once the piston 1138 has moved pass the pressure relief point 1139 and the pressure has abated. FIG. 12F shows the system 1100 after the spring 1144 has retracted the cartridge 1110 and other elements, thereby retracting the needle 1140 within the housing 1102.

The deployment spring 1134 and the retraction spring 1144 are configured (e.g., selected to have an appropriate spring rate) such that the entire process of activation, deployment, administration of the therapeutic agent, and retraction is performed within a predefined period of elapsed time. The predefined period of elapsed time is selected to be a clinically acceptable time period. In some embodiments, the period of elapsed time is between about 0.1 seconds and 2.0 seconds, including all values and sub-ranges therebetween, including, for example between about 0.1 sections and about 0.5 seconds.

While a spring-loaded piston system is described with reference to FIGS. 12A-12F, it can be appreciated that other forms of stored energy devices can be used. For example, instead of a spring 1134, an electric motor can be activated in response to actuation of the activation device 1136. Alternatively, a pressurized source of liquid and/or gas can be triggered to deliver pressurized liquid and/or gas in response to the actuation of the activation device 1136. In some embodiments, the stored energy device can act on an intermediary fluid, which then drives movement of a plunger, a cartridge, or other components of the delivery system, while in other embodiments, the stored energy device can act directly on a plunger, a cartridge, or other components of the delivery system.

While the throttle elements described herein are primarily described with reference to auto-injectors or automatic drug delivery devices, it can be appreciated that the throttle elements are not limited to auto-injector systems. For example, a fluidic throttle as described herein can be incorporated into any needle-based injector, e.g., to replace the needle as the restrictive orifice, including hand-held syringes. With a manually actuated syringe, the throttle can be affixed in the fluid path before the plunger applies the driving pressure to a therapeutic fluid. The throttle can then allow for constant drug extrusion from a manually applied force. Precise administration of therapy from prefilled syringes is critical to provide the highest level of care. Break-loose and glide forces of the driving plunger are carefully considered when developing these systems. The fluidic throttle, by delivering a constant force to the plunger, can allow for a uniform delivery of therapy. This is expected to reduce the need for chemical surface treatments for uniform glide forces. This is particularly useful in therapeutics that are sensitive to surface treatments during administration or storage.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100[units]” may mean within ±10% of 100 (e.g., from 90 to 110). The terms “about” and “approximately” may be used interchangeably.

“A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

1. An apparatus, comprising:
a needle having a proximal end and a distal end;
a cartridge having a body, a plunger, and a seal, the body, the plunger, and the seal collectively defining a reservoir configured to contain a therapeutic agent;
a stored energy device configured to apply pressure to an intermediary fluid;
a throttling assembly disposed upstream of the plunger in a pathway of the intermediary fluid; and
an activation device configured to activate the stored energy device to apply pressure to the intermediary fluid such that the intermediary fluid interacts with the throttling assembly to:
initially drive a movement of the cartridge and the needle to insert the distal end of the needle into tissue and the proximal end of the needle through the seal and into the reservoir; and
subsequently drive a movement of the plunger to extrude the therapeutic agent out through the needle.

2. The apparatus of claim 1, wherein the throttling assembly is configured to allow a rate of the insertion of the needle into the tissue to be greater or less than a rate of the extrusion of the therapeutic agent.

3. The apparatus of claim 1, wherein the throttling assembly includes an orifice configured to generate a throttled flow of the intermediary fluid downstream of the orifice in response to the intermediary fluid acting on the throttling assembly and flowing through the orifice.

4. The apparatus of claim 3, wherein the throttled flow of the intermediary fluid is a choked flow to generate a constant flow rate above a certain pressure difference between an inlet and an outlet of the orifice.

5. The apparatus of claim 3, wherein the orifice has a surface area of less than about 3 mm².

6. The apparatus of claim 1, wherein the throttling assembly is configured to reduce internal cartridge pressures that act on the cartridge in response to the activation of the stored energy device.

7. The apparatus of claim 1, wherein the intermediary fluid is liquid.

8. The apparatus of claim 1, further comprising a housing that contains the needle, the cartridge, the stored energy device, and the throttling assembly.

9. The apparatus of claim 1, further comprising a spring configured to retract the needle in response to a release in pressure being applied to the intermediary fluid.

10. The apparatus of claim 9, wherein the spring is a power spring, the stored energy device including a retraction spring configured to apply the pressure to the intermediary fluid, the retraction spring having a spring rate that is less than the spring rate of the power spring.

11. The apparatus of claim 1, wherein the throttling assembly is configured to prevent the needle from acting as a rate limiting restriction point for extruding the therapeutic agent.

12. The apparatus of claim 1, wherein the throttling assembly is configured to maintain an internal pressure of the cartridge at a predetermined pressure.

13. The apparatus of claim 1, wherein, in response to the activation of the stored energy device, the throttling assembly is configured to generate a flow of the intermediary fluid that applies a uniform pressure to the plunger to drive the movement of the plunger.

14. The apparatus of claim 1, wherein the throttling element is attached to a proximal end of the body of the cartridge.

15. An apparatus, comprising:
a cartridge having a plunger and a reservoir configured to contain a therapeutic agent;
a needle configured to extrude the therapeutic agent;
a stored energy device configured to apply pressure to an intermediary fluid;
a throttling assembly disposed upstream of the plunger in a pathway of the intermediary fluid; and
an activation device configured to activate the stored energy device to apply pressure to the intermediary fluid such that the throttling assembly generates a flow of the intermediary fluid that applies a constant pressure on the plunger to extrude the therapeutic agent through the needle.

16. The apparatus of claim 15, wherein the throttling assembly is configured to prevent the needle from acting as a rate limiting restriction point for extruding the therapeutic agent.

17. The apparatus of claim 15, wherein the throttling assembly is configured to maintain an internal pressure of the cartridge at a predetermined pressure.

18. The apparatus of claim 15, wherein the throttling assembly includes an orifice that is configured to throttle the flow of the intermediary fluid to affect the flow of the therapeutic agent.

19. The apparatus of claim 18, wherein the orifice has a diameter that is less than an inner diameter of the needle.

20. A method, comprising:
activating, by pressing an activation device, a stored energy device of a therapeutic delivery system;
in response to activating the stored energy device, applying pressure on an intermediary fluid to cause the intermediary fluid to interact with a throttling assembly of the therapeutic delivery system;
in response to the intermediary fluid interacting with the throttling element, driving movement of a cartridge and a needle of the therapeutic delivery system toward a skin of a patient to insert the needle into the skin;
in response to the intermediary fluid interacting with the throttling element, driving movement of the cartridge toward the needle such that the needle punctures through a seal of the cartridge to be in fluid communication with a therapeutic agent within the cartridge;
in response to the intermediary fluid interacting with the throttling element, generating a throttled flow of intermediary fluid via the throttling element; and
driving movement of a plunger of the cartridge to extrude an amount or volume of the therapeutic agent in response to the throttled flow of the intermediary fluid acting on the plunger.
